(19) United States
(12) Patent Application Publication
(10) Pub. No.: US 2011/0064686 A1
(43) Pub. Date: Mar. 17, 2011

(54) CATIONIC POLYMERS AS CONDITIONING AGENTS

(75) Inventors: Xiaodong Zhang, Livingston, NJ (US); Susan L. Jordan, Doylestown, PA (US); Tatiana V. Drovetskaya, Basking Ridge, NJ (US); Edward F. Diantonio, Staten Island, NY (US); Harry C. Silvis, Midland, MI (US)

(73) Assignee: DOW GLOBAL TECHNOLOGIES INC., Midland, MI (US)

(21) Appl. No.: 12/882,298

(22) Filed: Sep. 15, 2010

Related U.S. Application Data
(60) Provisional application No. 61/242,504, filed on Sep. 15, 2009.

(51) Int. Cl. A61K 8/81; C08F 220/06; A61K 8/891; A61Q 5/12

(52) U.S. Cl. 424/70.12; 526/318.6; 424/70.16

(57) ABSTRACT

The present invention provides personal care compositions comprising a cosmetically acceptable, cationically modified ethylene acrylic acid polymer that has a net cationic charge at pH 6, and a least one cosmetically acceptable surfactant, emollient, or cosmetic active.

### CROSS-REFERENCE TO RELATED APPLICATION(S)

This application claims benefit of priority from U.S. Provisional Patent Application No. 61/242,504, filed Sep. 15, 2009, which application is incorporated by reference herein to its entirety.

### FIELD

The present invention relates to personal care compositions.

### BACKGROUND

Conditioning of hair and/or skin is one of the most desired attributes in a personal care composition, particularly shampoos, body washes, and other personal care cleansers. Those skilled in the art are continually looking for conditioning agents that are more effective, more economical, or both.

Copolymerizing ethylene with acrylic acid yields ethylene-acrylic acid (EAA) copolymers, which are flexible thermoplastics. EAA finds use in blister packaging and adhesive lamination, particularly in food packaging applications. EAA is conventionally anionic, and hair and skin are negatively charged. As far as is known, those skilled in the art have hitherto found no use for EAA in personal care compositions.

### SUMMARY

In one embodiment, the present invention provides a cosmetically acceptable, cationically modified ethylene acrylic acid polymer that has a net cationic charge at pH 6.

In another embodiment, the present invention provides personal care compositions, comprising a cosmetically acceptable, cationically modified ethylene acrylic acid polymer that has a net cationic charge at pH 6, and a least one cosmetically acceptable surfactant, emollient, or cosmetic active.

### DETAILED DESCRIPTION

In one embodiment, the present invention provides a cosmetically acceptable, cationically modified ethylene acrylic acid polymer that has a net cationic charge at pH 6.

“Cosmetically acceptable” refers to ingredients typically used in personal care compositions, and is intended to underscore that materials that are toxic when present in the amounts typically found in personal care compositions are not contemplated as part of the present invention.

In one embodiment, the cationically modified ethylene acrylic acid polymer that has a net cationic charge at pH 6 is formed by reacting a secondary amine, tertiary amine, aromatic amine, an amino alkyl imidazole and/or an amidoamine with an ethylene/acrylic acid (EAA) copolymer.

Preferably, the EAA copolymer comprises greater than about 15 wt % acrylic acid, preferably greater than about 17 wt % acrylic acid, more preferably about 20 wt % acrylic acid. A preferred EAA copolymer is PRIMACOR 5990 copolymer (20 wt % acrylic acid), which has a melt index of 1300 g/10 minute (ASTM Method D-1238 at 190° C.) and a Brookfield viscosity of 13,000 cps at 350° F., and is available from The Dow Chemical Company. Another preferred EAA copolymer is PRIMACOR 5980i copolymer (20.5 wt % acrylic acid), which has a melt index of 300 g/10 minute (ASTM Method D-1238 at 190° C.), available from The Dow Chemical Company. EAA copolymers are also available under the tradename NUCREL 2806, available from E.I. du Pont de Nemours and Company, Inc. Ethylene-acrylic acid and ethylene-methacrylic acid copolymers, are described in U.S. Pat. Nos. 4,599,392, 4,988,781, and 5,938,437, each of which is incorporated herein by reference in its entirety.

Amidoamines are amides formed from fatty acids and diamines. In a preferred embodiment, amino alkyl imidazole is selected, a preferred amino alkyl imidazole being 1-(3-aminopropyl)-imidazole.

The cationically modified ethylene acrylic acid polymer that has a net cationic charge at pH 6 can be used in a variety of forms, for example, in powders or dispersions.

In one embodiment, the present invention provides personal care compositions, comprising a cosmetically acceptable, cationically modified ethylene acrylic acid polymer that has a net cationic charge at pH 6, and a least one cosmetically acceptable surfactant, emollient, or cosmetic active.

“Personal care” relates to compositions to be topically applied to a person (including mouth, ear, and nasal cavities, but not ingested). Examples of personal care compositions include skin care products (e.g., facial cream, moisturizers, leave on and rinse off lotions, sunscreens, foundation, mascara, eye-liner, lipstick, cleansers, and the like), oral care products (such as toothpastes and rinses), nail care products (such as polish and conditioners), and hair care products (including shampoos, leave on and rinse off conditioners, styling gels and hairsprays).

In one embodiment, the cationically modified ethylene acrylic acid polymer is present in a range from about 0.05 wt % to about 2.0 wt %, preferably from about 0.1 wt % to about 2.0 wt %, and more preferably from about 0.5 wt % to about 2.0 wt % of the personal care composition. This includes all subcombinations of ranges, as well as greater than 0.05 wt %, greater than 0.1 wt %, and greater than 0.5 wt %, and less than 2.0 wt %.

When present, the surfactant is an cationic, anionic, nonionic, or amphoteric surfactant, or a mixture thereof. In one embodiment, the surfactant is present in a range from about 1 wt % to about 30 wt %, preferably about 5 wt % to about 25 wt %, and more about 5 wt % to about 20 wt %.

In one embodiment, the anionic surfactant is ammonium laureth sulfate, ammonium lauryl sulfate, sodium laureth sulfate, or sodium lauryl sulfate.

Preferably, the mixture is an anionic surfactant in combination with an amphoteric surfactant. In one embodiment, the mixture is an anionic surfactant in combination with a second surfactant that is disodium cocoamphodiacetate, decylglucoside, or cocamidopropyl betaine.

In a preferred embodiment, the surfactant is a mixture of sodium laureth sulfate (such as is commercially available from Cognis as under the tradename STANDAPOL ES) and disodium cocoamphodiacetate (such as is commercially available from Henkel as under the tradename VELVETEX CDC). When the surfactant is a mixture of sodium laureth sulfate and disodium cocoamphodiacetate, the ratio of sodium laureth sulfate to disodium cocoamphodiacetate is in a range from about 15:1 to about 2:1, most preferably about 10:1.

In one embodiment, the composition includes a silicon based component, for example silicone oils, for instance volatile or non-volatile polymethylsiloxanes (PDMS) comprising a linear or cyclic silicone chain, which are liquid or pasty at room temperature, especially cyclopolydimethylsiloxanes (cyclomethicones) such as cyclopentasiloxane and cyclohexadimethylsiloxane, polydimethylsiloxanes comprising alkyl, alkoxy or phenyl groups, which are pendent or at the end of a silicone chain, these groups comprising from 2 to 24 carbon atoms, phenyl silicones, for instance phenyl trimethicones, phenyl dimethicones, phenyltrimethylsiloxydiphenylsiloxanes, diphenyl dimethicones, diphenylmethyldiphenyltrisiloxanes 2-phenylethyltrimethyl siloxysilicates and polymethylphenylsiloxanes, fluoro oils such as partially hydrocarbon-based and/or partially silicone-based fluoro oils, preferably dimethicone. A preferred blend of dimethicone, Laureth-23, and C 12-15 Pareth-3 is commercially available from Dow-Corning under the tradename DOW CORNING 2-1491 Silicone Emulsion, also described as a 60% large particle size non-ionic emulsion of a blend of ultra-high molecular weight polydimethylsiloxane gum and intermediate molecular weight polydimethylsiloxane fluid.

In one embodiment, the dimethicone is present in a range from about 0.1 wt % to about 5 wt %, preferably from about 0.75 wt % to about 3 wt %, and more preferably from about 1 wt % to about 2 wt %.

In one embodiment, the composition includes citric acid to adjust the pH.

In one embodiment, the composition includes at least one of cetearyl alcohol, cycloalkylsiloxane, or stearamidopropyl dimethylamine. In one embodiment, the cetearyl alcohol is present in a range from about 0.1 wt % to about 5 wt %, preferably from about 0.5 wt % to about 3 wt %, and more preferably from about 0.8 wt % to about 2.5 wt %. In one embodiment, the cycloalkylsiloxane is present in a range from about 0.1 wt % to about 5 wt %, preferably from about 0.5 wt % to about 3 wt %, and more preferably from about 0.8 wt % to about 2.5 wt %. In one embodiment, the stearamidopropyl dimethylamine is present in a range from about 0.1 wt % to about 5 wt %, preferably from about 0.5 wt % to about 3 wt %, and more preferably from about 0.8 wt % to about 2.5 wt %.

In one embodiment, the composition includes emollients, humectants, or other conditioning actives, for example, 2-pyrrolidone-5-carboxylic acid and its salts and esters, alkyl glucose alkoxylates or their esters, fatty alcohols, fatty esters, glycols and, in particular, methyl glucose ethoxylates or propoxylates and their stearate esters, isopropyl myristate, lanolin or cetyl alcohols, aloe, silicones, propylene glycol, glycerol and sorbitol.

Examples of emollients include hydrocarbon-based oils of animal origin, such as squalene, hydrocarbon-based oils of plant origin, such as liquid triglycerides of fatty acids comprising from 4 to 10 carbon atoms, for instance heptanoic or octanoic acid triglycerides, or alternatively, oils of plant origin, for example sunflower oil, corn oil, soybean oil, marrow oil, grapeseed oil, sesame seed oil, hazelnut oil, apricot oil, macadamia oil, arara oil, coriander oil, castor oil, avocado oil, jojoba oil, shea butter oil, or caprylic/capric acid triglycerides, MIGLYOL 810, 812 and 818 (commercially available from Dynamit Nobel), synthetic esters and ethers, especially of fatty acids, for instance the oils of formulae R¹COOR² and R¹OR² in which R¹ represents a fatty acid residue comprising from 8 to 29 carbon atoms and R² represents a branched or unbranched hydrocarbon-based chain comprising from 3 to 30 carbon atoms, for instance purcellin oil, isononyl isononanoate, isopropyl myristate, 2-ethylhexyl palmitate, 2-octyldodecyl stearate, 2-octyldodecyl erucate or isostearyl isostearate, hydroxylated esters, for instance isostearyl lactate, octyl hydroxystearate, octyldodecyl hydroxystearate, diisostearyl malate, triisocetyl citrate and fatty alcohol heptanoates, octanoates and decanoates, polyol esters, for instance propylene glycol dioctanoate, neopentyl glycol diheptanoate and diethylene glycol diisononanoate, pentaerythritol esters, for instance pentaerythrityl tetraisostearate, lipophilic derivatives of amino acids, such as isopropyl lauroyl sarcosinate, such as is sold under the name ELDEW SL 205 (commercially available from Ajinomoto), linear or branched hydrocarbons of mineral or synthetic origin, such as mineral oils (mixtures of petroleum-derived hydrocarbon-based oils), volatile or non-volatile liquid paraffins, and derivatives thereof, petroleum jelly, polydecenes, isohexadecane, isododecane, hydrogenated isoparaffin (or polyisobutene), ethers such as dicaprylyl ether (CTFA name: dicaprylyl ether), and C12-C15 fatty alcohol benzoates (such as FINSOLV TN from Finetex), mixtures thereof. Oils include mineral oil, lanolin oil, coconut oil and derivatives thereof, cocoa butter, olive oil, almond oil, macadamia nut oil, aloe extracts such as aloe vera lipoquinone, jojoba oils, safflower oil, corn oil, liquid lanolin, cottonseed oil, peanut oil, hydrogenated vegetable oil, squalane, castor oil, polybutene, sweet almond oil, avocado oil, calophyllum oil, ricin oil, vitamin E acetate, olive oil, silicone oils such as dimethylpolysiloxane and cyclomethicone, linolenic alcohol, oleyl alcohol, and the oil of cereal germs.

Other suitable emollients include dicaprylyl ether, C12-15 alkyl benzoate, DC 200 FLUID 350 silicone fluid (commercially available from Dow Corning Corp.), isopropyl palmitate, octyl palmitate, isopropyl myristate, hexadecyl stearate, butyl stearate, decyl oleate, acetyl glycerides, the octanoates and benzoates of C12-15 alcohols, the octanoates and decanoates of alcohols and polyalcohols such as those of glycol and glyceryl, ricinolates esters such as isopropyl adipate, hexyl laurate and octyl dodecanoate, dicaprylyl maleate, phenyltrimethicone, and aloe vera extract. Solid or semi-solid cosmetic emollients include glyceryl dilaurate, hydrogenated lanolin, hydroxylated lanolin, acetylated lanolin, petrolatum, isopropyl lanolate, butyl myristate, cetyl myristate, myristyl myristate, myristyl lactate, cetyl alcohol, isostearyl alcohol and isocetyl lanolate. In a preferred embodiment, the emollient is at least one of myristates, stearates, palmitates, cocoates, coco-caprylate/caprates, oleates, hexyldecanol, octyldodecanol, caprylic/capric triglycerides, or cocoglycerides, and their alkoxylates.

Contemplated conditioning agents include stearalkonium chloride, dicetyldimonium chloride, lauryl methyl gluceth-10 hydroxypropyldimonium chloride, and conditioning polymers such as polyquarternium-10, polyquarternium-24 and chitosan and derivatives thereof.

Other optional ingredients for personal care compositions of the present invention include cosmetically acceptable emollients, sunscreens, surfactants, emulsifiers, preservatives, rheology modifiers, colorants, preservatives, pH adjustors, propellants, reducing agents, fragrances, foaming agents, tanning agents, depilatory agents, flavors, astringents, antiseptics, deodorants, antiperspirants, insect repellants, bleaches, lighteners, anti-dandruff agents, adhesives, polishes, strengtheners, fillers, barrier materials, or biocides.

In some embodiments, the personal care composition further comprises an optional rheology modifier as a thickener. Examples of thickeners include polymers, for example, modified or unmodified carboxyvinyl polymers, such as the products sold under the names CARBOPOL and PEMULEN (INCI name: Acrylates/C₁₀₋₃₀ alkyl acrylate crosspolymer; available from Noveon), polyacrylates and polymethacrylates, such as the products sold under the names LUBRAJEL and NORGEL (commercially available from Guardian) or HISPAGEL (commercially available from Hispano Chimica), polyacrylamides, 2-acrylamido-2-methylpropanesulfonic acid polymers and polymers, which are optionally crosslinked and/or neutralized, for instance the poly(2-acrylamido-2-methylpropane-sulfonic acid) sold by Clariant (INCI name: ammonium polyacryldimethyltauramide), emulsified crosslinked anionic polymers of acrylamide and AMPS, such as those sold under the name SEPIGEL 305 (INCI name: Polyacrylamide/C13-14 Isoparaffin/Laureth-7; from Seppic) and under the name SIMULGEL 600 (INCI name: Acrylamide/Sodium acryloyldimethyltaurate polymer/Isohexadecane/Polysorbate 80; from Seppic), polysaccharide biopolymers, for instance xanthan gum, guar gum, carob gum, acacia gum, scleroglucans, chitin and chitosan derivatives, carrageenans, gellans, alginates, celluloses such as microcrystalline cellulose, cellulose derivatives, associative polymers, for instance associative polyurethanes, polymers comprising at least two hydrocarbon-based lipophilic chains comprising from 6 to 30 carbon atoms, separated with a hydrophilic sequence, such as the polyurethanes sold under the names SERAD FX1010, SERAD FX1100 and SERAD FX1035 (commercially available from Hüls America), RHEOLATE 255, RHEOLATE 278 and RHEOLATE 244 (INCI name: Polyether-urea-polyurethane; from Rheox), DW 1206F, DW 1206J, DW 1206B, DW 1206G, and ACRYSOL RM 2020 (commercially available from Rohm & Haas).

Colorants include pigments, which are used especially in make-up, including metal oxide pigments, titanium dioxide, optionally surface-treated, zirconium oxide or cerium oxide, zinc oxide, iron oxide (black, yellow or red), chromium oxide, manganese violet, ultramarine blue, chromium hydrate and ferric blue, carbon black, pigments of barium, strontium, calcium or aluminum (for example D&C or FD&C), cochineal carmine, mica coated with titanium or with bismuth oxychloride, titanium mica with iron oxides, titanium mica with, especially, ferric blue or chromium oxide, titanium mica with an organic pigment, nacreous pigments based on bismuth oxychloride, goniochromatic pigments, for example pigments with a multilayer interference structure, reflective pigments, for example particles with a silver-coated glass substrate, glass substrate coated with nickel/chromium/molybdenum alloy, glass substrate coated with brown iron oxide, particles comprising a stack of at least two polymer layers, for instance MIRROR GLITTER (commercially available from 3M).

Dyes include water-soluble dyes such as copper sulfate, iron sulfate, water-soluble sulfopolyesters, rhodamines, natural dyes, for instance carotene and beetroot juice, methylene blue, caramel, the disodium salt of tartrazine and the disodium salt of fuschin, and mixtures thereof. Liposoluble dyes from the list above may also optionally be used.

Preservatives include alcohols, aldehydes, methylchloroisothiazolinone and methylisothiazolinone, p-hydroxybenzoates, and in particular methylparaben, propylparaben, glutaraldehyde and ethyl alcohol.

The pH adjustors, include inorganic and organic acids and bases and in particular aqueous ammonia, citric acid, phosphoric acid, acetic acid, and sodium hydroxide.

Reducing agents include ammonium thioglycolate, hydroquinone and sodium thioglycolate.

Fragrances may be aldehydes, ketones, or oils obtained by extraction of natural substances or synthetically produced as described above. Often, fragrances are accompanied by auxiliary materials, such as fixatives, extenders, stabilizers and solvents.

Biocides include antimicrobials, bactericides, fungicides, algaecides, mildicides, disinfectants, antiseptics, and insecticides.

The amount of optional ingredients effective for achieving the desired property provided by such ingredients can be readily determined by one skilled in the art.

In a preferred embodiment, the personal care composition is a shampoo, body wash, or facial cleanser, preferably a shampoo.

In another preferred embodiment, the personal care composition is a rinse off hair conditioner. Such a conditioner may further comprise at least one fatty alcohol, fatty amine, cationic monoquat, polyquat, silicone, or other oil.

In another preferred embodiment, the personal care composition is a leave on hair conditioner.

In yet another preferred embodiment, the personal care composition is a skin conditioner.

### EXAMPLES

The following examples are for illustrative purposes only and are not intended to limit the scope of the present invention. All percentages are by weight unless otherwise specified.

### Example 1

Exemplary personal care compositions of the present invention contain the components recited in TABLE 1 to form a shampoo.

TABLE 1

| Component | Batch 1 | Batch 2 |
|---|---|---|
| STANDAPOL ES-2 Sodium Laureth Sulfate | 60.78 | 60.78 |
| VELVETEX CDC Disodium | 6.92 | 6.92 |
| Cocoamphodiacetate | | |
| Cationically Modified Ethylene Acrylic Acid | 0.5 | 0.5 |
| Polymer | | |
| DOW CORNING 2-1491 Silicone Emulsion | 0 | 1.67 |
| (Dimethicone, Laureth-23, and C 12-15 | | |
| Pareth-3) | | |
| Citric Acid | 2.1 | 2.1 |
| GLYDANT DMDM Hydantoin | 0.4 | 0.4 |
| LEXEMUL EDGS pearlizing agent | 2.0 | 2.0 |
| DI Water | QS to 100 | QS to 100 |

The cationically modified ethylene acrylic acid polymer is made according to the following protocol. PRIMACOR 5980 (20 wt % acrylic acid, 278 mmol AA) and 1-(3-aminopropyl)-imidazole) (883 mmol, 4× molar excess) are combined under nitrogen in a three neck round bottom flask with an outlet for the purge passing through a reflux condenser. As a precaution in case of loss of nitrogen flow, the outlet is trapped by a bubbler to prevent oxygen diffusion. The flow rate of oxygen must be sufficient to quickly remove water by-products.

The flask is submerged in an oil bath at about 170° C. for 48 hrs. At 48 hrs, the melt should appear transparent and slightly yellow (brown indicates undesirable oxidative degradation). The melt is poured into a pan to cool, then washed with acetone to remove unreacted imidazole. The material may be cut, washed again with acetone, dried and then ground to a fine powder. The powder is washed with acetone and/or stirred in acetone to remove any residual imidazole.

To make the personal care compositions, sodium laureth sulfate and disodium cocoamphodiacetate are combined and heated to 75° C. with slow agitation (approximately 500 rpm) to form a surfactant solution. Pearlizing agent is added at approximately 500 rpm, maintaining speed for 15 minutes. The mixture is slowly cooled to about 35° C. If present, the silicone emulsion is added and allowed to disperse with stirrer speed about 750 rpm, for about 15 minutes. Then, the cationic polymer is slowly added and allowed to totally disperse (about 30 minutes). Citric acid is added to adjust pH and preservative is also added. The resulting mixture is cooled to 25° C., and the formulation is adjusted to 100% with DI water.

### Example 2

### Comparative

Exemplary comparative personal care compositions contain the components recited in TABLE 2.

TABLE 2

| | Batch A | Batch B |
|---|---|---|
| Component | (Comparative) | (Comparative) |
| STANDAPOL ES-2 Sodium Laureth | 60.78 | 60.78 |
| Sulfate | | |
| VELVETEX CDC Disodium | 6.92 | 6.92 |
| Cocoamphodiacetate | | |
| UCARE JR-400 Polyquaternium-10 | 25 | 25 |
| conditioner (2% solution) | | |
| DOW CORNING 2-1491 Silicone | 0 | 1.67 |
| Emulsion (Dimethicone, Laureth-23, | | |
| and C 12-15 Pareth-3) | | |
| Citric Acid | 2.1 | 2.1 |
| GLYDANT DMDM Hydantoin | 0.4 | 0.4 |
| LEXEMUL EDGS pearlizing agent | 2.0 | 2.0 |
| DI Water | QS to 100 | QS to 100 |

The formulations are prepared in a manner substantially similar to that of Example 1. UCARE JR-400 Polyquaternium-10 conditioner is a leading conditioning ingredient.

### Example 3

Formulations made substantially according to the protocols described above in Examples 1 and 2 were made and tested as recited in Tables 3A and 3B:

TABLE 3A

| | Wet Feel | Wet Comb | Dry Feel | Dry Comb |
|---|---|---|---|---|
| Batch 1 | 15 | 15 | 12 | 12 |
| Batch A | 5 | 5 | 8 | 8 |
| (Comparative) | | | | |

TABLE 3B

| | Wet Feel | Wet Comb | Dry Feel | Dry Comb |
|---|---|---|---|---|
| Batch 2 | 14 | 14 | 15 | 15 |
| Batch B | 6 | 6 | 5 | 5 |
| (Comparative) | | | | |

A panel of 20 persons is asked to feel and comb the tresses in both wet and dry condition to determine wet and dry feel preference and wet and dry combability. For each panelist, two pre-washed and pre-hydrated tresses of European virgin brown hair, commercially available from International Hair Importers and Products Inc. NY (USA), are compared, one hair tress treated with 0.5 g of one composition, the other hair tress treated with 0.5 g of the other composition, as listed in TABLES 3A & 3B. The compositions are worked into the hair for one minute, then rinsed off under running tap water at ˜38° C. (about 0.4 gal/min). Each panelist is asked to compare the tresses and state which tress is smoother to comb/feel. The answer “same” is not allowed. The reported number is the number of panelists preferring one over the other.

Compositions of the present invention showed significantly better feel and combability, both wet and dry.

### Example 4

Exemplary personal care compositions of the present invention contain the components recited in TABLE 4 to form a rinse off conditioner.

TABLE 4

| | Component | Batch 3 |
|---|---|---|
| A | PROMULGEN D Emulsifier | 2.5 |
| | DOW CORNING 345Fluid (blend of | 1.0 |
| | polydimethylcyclosiloxane composed mainly of | |
| | cyclopentasiloxane and cyclohexasiloxane) | |
| | JEECHEM S-13 Stearamidopropyl Dimethylamine | 0.8 |
| | Stearyl Alcohol | 1.5 |
| | Cationically Modified Ethylene Acrylic Acid Polymer | 2.0 |
| B | DI Water | 90.8 |
| | GLUCAM E-10 humectant | 1.0 |
| C | GLYDANT DMDM Hydantoin | 0.4 |

Phase A ingredients were combined, mixed, and heated to 75° C. under stiffing. In a separate reaction vessel, phase B ingredients were combined, mixed and heated to 75° C. under stiffing. Phases A and B are combined at 75° C. under stirring at 470 rpm. The reaction mixture is allowed to cool to room temperature while stiffing. Phase C is added at 35° C. The pH of the solution is decreased to about 4 to about 5 using 50% citric acid as needed.

### Example 5

### Comparative

Exemplary comparative personal care compositions contain the components recited in TABLE 5.

TABLE 5

| Component | Batch C (Comparative) |
|---|---|
| PROMULGEN D Emulsifier | 2.5 |
| DOW CORNING 345Fluid (blend of | 1.0 |
| polydimethylcyclosiloxane composed mainly of | |
| cyclopentasiloxane and cyclohexasiloxane) | |
| JEECHEM S-13 Stearamidopropyl | 0.8 |
| Dimethylamine | |
| Stearyl Alcohol | 1.5 |
| MAQUAT BTMC-85 Behentrimonium chloride | 2.0 |
| cationic monoquat | |
| GLUCAM E-10 humectant | 1.0 |
| Citric Acid | 0.21 |
| GLYDANT DMDM Hydantoin | 0.4 |
| DI Water | QS to 100 |

The formulations are prepared in a manner substantially similar to that of Example 4. MAQUAT BTMC-85 Behentrimonium chloride is a monoquat/cationic surfactant commonly used in rinse-off conditioner products to achieve conditioning performance.

### Example 6

Formulations made substantially according to the protocols described above in Examples 4 and 5 were made and tested as recited in Table 6:

TABLE 6

| | Dry Feel | Dry Comb |
|---|---|---|
| Batch 3 | 10 | 9 |
| Batch C | 0 | 1 |
| (Comparative) | | |

A panel of ten expert panelists trained to evaluate performance of cosmetic products on hair were asked to evaluate combality and feel. For each panelist, two pre-washed and pre-hydrated tresses of European virgin brown hair, commercially available from International Hair Importers and Products Inc. NY (USA), are compared, one hair tress treated with 0.5 g of one composition, the other hair tress treated with 0.5 g of the other composition, as listed in TABLE 6. The compositions are worked into the hair for one minute, then rinsed off under running tap water at ˜38° C. (about 0.4 gal/min). The tresses were hung to dry over night. Each panelist is asked to compare the tresses and state which tress combed easier and which tress felt silkier, softer, and smoother. The answer “same” is not allowed. The reported number is the number of panelists preferring one over the other.

Compositions of the present invention showed significantly better feel and combability than a leading monoquat conditioning ingredient.

### Example 7

Dispersion of Cationically Modified Ethylene Acrylic Acid Polymer is prepared as follows: PRIMACOR 5980i (100 g, 0.278 mol acrylic acid) and 1-(3-aminopropyl) imidazole (104.3 g, 0.833 mol) are added to a 500 mL, three neck, round bottom flask equipped with an overhead stir shaft, a reflux condenser, and a nitrogen inlet. The flask is immersed in a silicon oil bath at 180° C. The mixture is stirred for 2 days. After such time, the slightly yellow viscous solution is poured into a pyrex crystallizing dish followed by cooling to the ambient temperature. The polymer is then cryogenically ground into powder and washed with excess acetone overnight. The polymer is filtered and dried in vacuo at 35° for a day. The polymer is pressed into a film at 140° C. and 20,000 psi. Substantially following this protocol, the ¹H NMR (C2D2C14) at 80° C. was obtained and the conversion of acrylic acid to amidopropyl imidazole was calculated to be 82.5%.

30.1 g of amidopropyl imidazole substituted polymer, phosphoric acid (7.24 g), and water are placed in a Parr reactor vessel fitted with a Cowles blade. The material is heated to 125° C. while mixing slowly. Once the set temperature is reached, the mixer is run on high (˜1800 rpm) for 25 minutes. Heat is removed and stirring continues until the temperature cools to at least 30° C. The final aqueous dispersion is then collected.

It is understood that the present invention is not limited to the embodiments specifically disclosed and exemplified herein. Various modifications of the invention will be apparent to those skilled in the art. Such changes and modifications may be made without departing from the scope of the appended claims.

Moreover, each recited range includes all combinations and subcombinations of ranges, as well as specific numerals contained therein. Additionally, the disclosures of each patent, patent application, and publication cited or described in this document are hereby incorporated herein by reference, in their entireties.

1. A cosmetically acceptable, cationically modified ethylene acrylic acid polymer that has a net cationic charge at pH 6.

2. A personal care composition, comprising:
the polymer of claim 1, and
a least one cosmetically acceptable surfactant, emollient, or cosmetic active.

3. The personal care composition of claim 2, wherein the cationically modified ethylene acrylic acid polymer is formed from an ethylene/acrylic acid copolymer comprising greater than about 15 wt % acrylic acid, preferably greater than about 17 wt % acrylic acid, more preferably about 20 wt % acrylic acid.

4. The personal care composition of claim 2, wherein the cationically modified ethylene acrylic acid polymer is formed from at least one of an amino alkyl imidazole or an amidoamine.

5. The personal care composition of claim 2, wherein the cationically modified ethylene acrylic acid polymer is formed from an ethylene/acrylic acid copolymer comprising about 20 wt % acrylic acid and a 1-(3-aminopropyl)-imidazole.

6. The personal care composition of claim 2, wherein the cationically modified ethylene acrylic acid polymer is present in a range from about 0.05 wt % to about 2.0 wt %, preferably from about 0.1 wt % to about 2.0 wt %, and more preferably from about 0.5 wt % to about 2.0 wt %.

7. The personal care composition of claim 2, wherein the surfactant is present, and the personal care composition is a shampoo.

8. The personal care composition of claim 2, wherein the personal care composition is a conditioner.

9. The personal care composition of claim 7, wherein sodium laureth sulfate and disodium cocoamphodiacetate are present and wherein the cationically modified ethylene acrylic acid polymer is present in a range from about 0.05 wt % to about 2.0 wt %.

10. The personal care composition of claim 7 or 9, further comprising dimethicone.

11. The personal care composition of claim 10, wherein the dimethicone is present in a range from about 0.1 wt % to about 5 wt %, preferably from about 0.75 wt % to about 3 wt %, and more preferably from about 1 wt % to about 2 wt %.

12. The personal care composition of claim 2 or 8, comprising at least one of cetearyl alcohol, cycloalkylsiloxane, or stearamidopropyl dimethylamine.

13. The personal care composition of claim 2, wherein the cosmetically acceptable, cationically modified ethylene acrylic acid polymer is in the form of an aqueous dispersion.
